(19) United States
(12) Patent Application Publication
(10) Pub. No.: US 2025/0182881 A1
(43) Pub. Date: Jun. 5, 2025

(54) MEDICAL IMAGE PROCESSING APPARATUS AND X-RAY COMPUTED TOMOGRAPHY APPARATUS

(71) Applicant: Canon Medical Systems Corporation, Otawara-shi (JP)

(72) Inventors: Shinsuke TSUKAGOSHI, Nasushiobara (JP); Yohei MINATOYA, Tokyo (JP)

(73) Assignee: Canon Medical Systems Corporation, Otawara-shi (JP)

(21) Appl. No.: 18/969,248

(22) Filed: Dec. 4, 2024

(30) Foreign Application Priority Data
Dec. 5, 2023 (JP) 2023-205311

(51) Int. Cl. G16H 30/20; A61B 6/00; A61B 6/03; A61B 6/42; A61B 6/46

(52) U.S. Cl. CPC G16H 30/20; A61B 6/032; A61B 6/463; A61B 6/5294; A61B 6/4241

(57) ABSTRACT

The processing circuitry acquires examination information related to a PCCT scan, the examination information including generation purpose information indicating a generation purpose of an image and generation process information indicating a generation process of the image according to the generation purpose. The processing circuitry generates, according to the generation process, a PCCT image according to the generation purpose based on count data collected by the PCCT scan. The processing circuitry stores the examination information including the generation purpose information and the generation process information in a storage device in association with the PCCT image.

### CROSS-REFERENCE TO RELATED APPLICATIONS

This application is based upon and claims the benefit of priority from Japanese Patent Application No. 2023-205311, filed Dec. 5, 2023, the entire contents of which are incorporated herein by reference.

### FIELD

Embodiments described herein relate generally to medical image processing apparatus and an X-ray computed tomography apparatus.

### BACKGROUND

Photon counting computed tomography (PCCT) scans can use multiple energy bins to generate a wide variety of PCCT images or identify substances. Since there are a wide variety of generation processes for generating a PCCT image for similar purposes, it is difficult to determine the generation purpose and generation process of the PCCT image only by observing and confirming the obtained PCCT image and the scan conditions thereof.

### BRIEF DESCRIPTION OF THE DRAWINGS

FIG. 1 is a diagram showing a configuration of an X-ray computed tomography apparatus according to the present embodiment.

FIG. 2 is a diagram showing an example of setting the number of energy bins and an energy range.

FIG. 3 is a diagram showing details of a process of generating a PCCT image.

FIG. 4 is a diagram showing a specific example of a process of generating a counting mode.

FIG. 5 is a view showing a process procedure of a PCCT examination by the X-ray computed tomography apparatus.

FIG. 6 is a diagram showing a display example of a PCCT image with a generation purpose “equivalent to 75 keV” according to pattern 1 and various kinds of examination information.

FIG. 7 is a diagram showing a display example of a PCCT image with a generation purpose “equivalent to 75 keV” according to pattern 2 and various kinds of examination information.

FIG. 8 is a diagram showing a display example of a PCCT image of a generation purpose “material decomposition (iodine & calcium)” and various kinds of examination information.

FIG. 9 is a conceptual diagram of association between a PCCT image and various kinds of examination information.

FIG. 10 is a diagram showing a first example of input of various kinds of examination information to a DICOM standard tag.

FIG. 11 is a diagram showing a second example of input of various kinds of examination information to the DICOM standard tag.

FIG. 12 is a diagram showing a process procedure of display processing of a PCCT image by a medical image processing apparatus according to a first modification.

### DETAILED DESCRIPTION

In general, according to one embodiment, a medical image processing apparatus and X-ray computed tomography apparatus include an acquisition unit, a generation unit, and a storage unit. The acquisition unit acquires examination information that is related to the PCCT scan and includes generation purpose information indicating a generation purpose of an image and generation process information indicating a generation process of an image according to the generation purpose. The generation unit generates, according to the generation process, a PCCT image according to the generation purpose based on the count data collected by the PCCT scan. The storage unit stores the examination information including the generation purpose information and the generation process information in a storage device in association with the PCCT image.

Hereinafter, the medical image processing apparatus and the X-ray computed tomography apparatus according to the present embodiment will be described in detail with reference to the drawings.

The X-ray computed tomography apparatus according to the present embodiment includes various types such as a third generation CT and a fourth generation CT, and any type is applicable to the present embodiment. Here, the third generation CT is a Rotate/Rotate-Type in which the X-ray tube and the X-ray detector integrally rotate around the subject. The fourth generation CT is a Stationary/Rotate-Type in which a large number of X-ray detection elements arranged in a ring shape are fixed, and only an X-ray tube rotates around a subject. In addition, the X-ray computed tomography apparatus according to the present embodiment can be applied to a single tube type in which one pair of an X-ray tube and an X-ray detector is mounted on a rotation ring, or a multi-tube type in which a plurality of pairs of an X-ray tube and an X-ray detector is mounted on a rotation ring. However, in the following description, the X-ray computed tomography apparatus is assumed to be a single tube type.

The X-ray computed tomography apparatus according to the present embodiment is assumed to be a photon counting CT apparatus that performs photon counting computed tomography (PCCT).

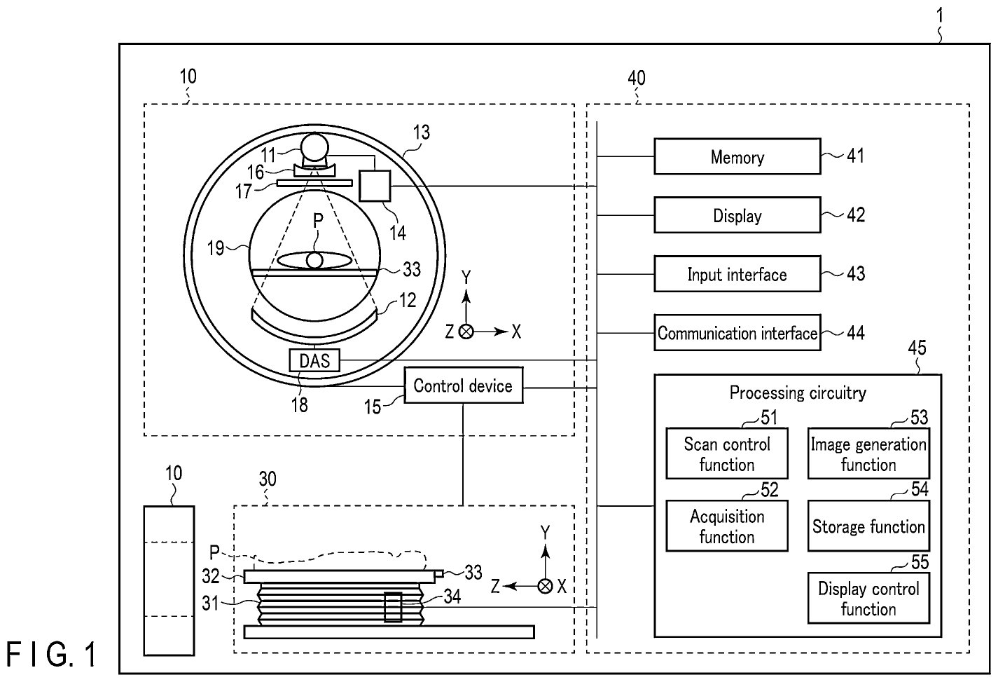

FIG. 1 is a diagram showing a configuration of X-ray computed tomography apparatus 1 according to the present embodiment. As shown in FIG. 1, the X-ray computed tomography apparatus 1 includes a gantry 10, a couch 30, and a console 40. Note that, in FIG. 1, the gantry 10 is shown at a plurality of locations for convenience of description, but one stand 10 or a plurality of stands may be mounted on the X-ray computed tomography apparatus 1. The gantry 10 is a scanning device having a configuration for PCCT scanning of a subject P. The couch 30 is a transfer device for placing the subject P to be subjected to the X-ray CT imaging and positioning the subject P. The console 40 is a computer that controls the gantry 10. For example, the gantry 10 and the couch 30 are installed in a CT examination room, and the console 40 is installed in a control room adjacent to the CT examination room. The gantry 10, the couch 30, and the console 40 are connected in a wired or wireless manner so as to be able to communicate with each other. The console 40 is not necessarily installed in the control room. For example, the console 40 may be installed in the same room together with the gantry 10 and the couch 30. The console 40 may also be incorporated into the gantry 10. The console 40 is an example of the medical image processing apparatus.

As shown in FIG. 1, the gantry 10 includes an X-ray tube 11, an X-ray detector 12, a rotary frame 13, an X-ray high-voltage device 14, a control device 15, a wedge 16, a collimator 17, and a data acquisition system (DAS) 18.

The X-ray tube 11 generates an X-ray. Specifically, the X-ray tube 11 includes a cathode that generates thermoelectrons, an anode that receives the thermoelectrons flying from the cathode and generates X-rays, and a vacuum tube that holds the cathode and the anode. The X-ray tube 11 is connected to the X-ray high-voltage device 14 through a high voltage cable. A tube voltage is applied between the cathode and the anode by the X-ray high-voltage device 14. The application of the tube voltage causes thermoelectrons to fly from the cathode toward the anode. If thermoelectrons fly from the cathode toward the anode, a tube current flows. By application of a high voltage and supply of a filament current from the X-ray high-voltage device 14, thermoelectrons fly from the cathode (filament) toward the anode (target), and the thermoelectrons collide with the anode, thereby generating X-rays. For example, the X-ray tube 11 is a rotating anode type X-ray tube that generates X-rays by irradiating a rotating anode with thermoelectrons.

The X-ray detector 12 detects the X-rays generated from the X-ray tube 11 and passing through the subject P, and outputs an electric signal corresponding to the energy of the detected X-rays to the data acquisition system 18. The X-ray detector 12 has a structure in which a plurality of X-ray detection element arrays in which a plurality of X-ray detection elements are arrayed in the channel direction are arrayed in the slice direction (column direction). The X-ray detector 12 is, for example, an indirect conversion type detector having a grid, a scintillator array, and an optical sensor array. The scintillator array includes a plurality of scintillators. The scintillator generates a plurality of fluorescent photons according to the energy of incident X-ray photons. The grid includes an X-ray shielding plate that is disposed on the X-ray incident surface side of the scintillator array and absorbs scattered X-rays. Note that the grid may also be referred to as a collimator (a one-dimensional collimator or two-dimensional collimator). The optical sensor array converts the plurality of fluorescent photons from the scintillator into an electrical signal having a peak value corresponding to the energy of the incident X-ray photons. As the optical sensor, for example, a photodiode is used.

The X-ray detector 12 may be a direct-conversion type detector. As the direct-conversion type X-ray detector 12, for example, a type including a semiconductor diode in which electrodes are attached to both ends of a semiconductor is applicable. The X-ray photons incident on the semiconductor are converted into electron-hole pairs. The number of electron-hole pairs generated by the incidence of one X-ray photon depends on the energy of the incident X-ray photon. The electrons and the holes are attracted to each other by a pair of electrodes formed at both ends of the semiconductor. The pair of electrons generate electrical signals having a peak value corresponding to the charge of the electron-hole pairs. One electrical signal includes a peak value corresponding to the energy of the incident X-ray photon.

The rotary frame 13 is an annular frame that rotatably supports the X-ray tube 11 and the X-ray detector 12 about a rotation axis (Z axis). Specifically, the rotary frame 13 supports the X-ray tube 11 and the X-ray detector 12 facing each other. The rotary frame 13 is supported by a fixed frame (not shown) so as to be rotatable about a rotation axis. The control device 15 rotates the rotary frame 13 about the rotation axis to rotate the X-ray tube 11 and the X-ray detector 12 about the rotation axis. The rotary frame 13 receives power from the drive mechanism of the control device 15 and rotates around the rotation axis at a constant angular velocity. An image field of view (FOV) is set in an opening 19 of the rotary frame 13.

In the present embodiment, the longitudinal direction of the rotation axis of the rotary frame 13 or a top plate 33 of the couch 30 in the non-tilting state is defined as the Z-axis direction, the axial direction orthogonal to the Z-axis direction and horizontal to the floor surface is defined as the X-axis direction, and the axial direction orthogonal to the Z-axis direction and vertical to the floor surface is defined as the Y-axis direction.

The X-ray high-voltage device 14 includes a high-voltage generator and an X-ray controller. The high voltage generator includes an electric circuit such as a transformer and a rectifier, and generates a high voltage to be applied to the X-ray tube 11 and a filament current to be supplied to the X-ray tube 11. The X-ray control device controls an output voltage according to the X-rays emitted from the X-ray tube 11. The high-voltage generator may be a transformer type or an inverter type. The X-ray high-voltage device 14 may be provided in the rotary frame 13 in the gantry 10, or may be provided in a fixed frame (not shown) in the gantry 10.

The wedge 16 adjusts the dose of the X-rays with which the subject P is irradiated. Specifically, the wedge 16 attenuates the X-rays so that the dose of the X-rays emitted from the X-ray tube 11 to the subject P has a predetermined distribution. For example, a metal plate made of aluminum or the like such as a wedge filter or a bow-tie filter is used as the wedge 16.

The collimator 17 limits an irradiation range of the X-rays transmitted through the wedge 16. The collimator 17 slidably supports a plurality of lead plates that shield X-rays, and adjusts a form of a slit formed by the plurality of lead plates. The collimator 17 may also be referred to as an X-ray aperture.

The data acquisition system 18 collects count data of X-rays detected by the X-ray detector 12 for each energy bin. As an example, the data acquisition system 18 includes a pre-amplifier, a waveform shaping circuit, a pulse-height discriminator circuit, and a counting circuit. The pre-amplifier amplifies an electric signal having a peak value corresponding to the energy of the X-ray photon detected by the X-ray detector 12 at a predetermined magnification. The waveform shaping circuit shapes the waveform of the electrical signal output by the pre-amplifier. The pulse-height discriminator applies an energy threshold corresponding to each of the plurality of energy bins to the electrical signal output by the waveform shaping circuit, and outputs an electrical pulse signal corresponding to the energy bin to which the electrical signal belongs. The counting circuit counts the electrical pulse signal output from the pulse-height discriminator in units of view periods for each energy bin, thereby generating count data representing the number of the count of X-ray photons for each energy bin. The data acquisition system 18 is implemented by, for example, an application specific integrated circuit (ASIC). The count data is transmitted to the console 40 via a non-contact data transmission device or the like.

The control device 15 controls the X-ray high-voltage device 14 and the data acquisition system 18 to perform a PCCT scan according to the control of a processing circuitry 45 of the console 40. The control device 15 includes a processing circuitry including a central processing unit (CPU), a micro processing unit (MPU), or the like, and a drive mechanism such as a motor and an actuator. The processing circuitry includes a processor such as a CPU and a memory such as a read only memory (ROM) and a random access memory (RAN) as hardware resources. Furthermore, the control device 15 may be implemented by an ASIC or a field programmable gate array (FPGA). Furthermore, the control device 15 may be implemented by another complex programmable logic device (CPLD) or a simple programmable logic device (SPLD). The control device 15 has a function of controlling the operation of the gantry 10 and the couch 30 in response to an input signal from an input interface 43 (described later) attached to the console 40 or the gantry 10. For example, the control device 15 performs control to rotate the rotary frame 13 in response to an input signal, control to tilt the gantry 10, and control to operate the couch 30 and the top plate 33. Note that the control of tilting the gantry 10 is implemented by the control device 15 rotating the rotary frame 13 about an axis parallel to the X-axis direction based on the inclination angle (tilt angle) information input by the input interface attached to the gantry 10. Note that the control device 15 may be provided on the gantry 10 or may be provided on the console 40.

The couch 30 includes a base 31, a support frame 32, a top plate 33, and a couch driving device 34. The base 31 is installed on a floor surface. The base 31 is a housing that supports the support frame 32 so as to be movable in a direction perpendicular to the floor surface (Y-axis direction). The support frame 32 is a frame provided above the base 31. The support frame 32 slidably supports the top plate 33 along the rotation axis (Z axis). The top plate 33 is a flexible plate on which the subject P is placed.

The couch driving device 34 is accommodated in a housing of the couch 30. The couch driving device 34 is a motor or an actuator that generates power for moving the support frame 32 and the top plate 33 on which the subject P is placed. The couch driving device 34 operates according to control by the console 40 or the like.

The console 40 includes a memory 41, a display 42, an input interface 43, a communication interface 44, and a processing circuitry 45. Data communication among the memory 41, the display 42, the input interface 43, the communication interface 44, and the processing circuitry 45 is performed via a bus (BUS). Although the console 40 will be described as a separate body from the gantry 10, the gantry 10 may include a part of each component of the console 40 or the console 40.

The memory 41 is a storage device such as a hard disk drive (HDD), a solid state drive (SSD), or an integrated circuit storage device that stores various types of information. The memory 41 stores, for example, count data and PCCT image data. The memory 41 may be a portable storage medium such as a compact disc (CD), a digital versatile disc (DVD), or a flash memory, in addition to an HDD, an SSD, or the like. The memory 41 may be a drive device that reads and writes various types of information from and to a semiconductor memory element such as a flash memory or a random access memory (RAN). In addition, the storage area of the memory 41 may be in the X-ray computed tomography apparatus 1 or in an external storage device connected via a network. The memory 41 stores a database to be described later.

The display 42 displays various types of information. For example, the display 42 outputs the PCCT image generated by the processing circuitry 45, a graphical user interface (GUI) for receiving various operations from the operator, and the like. As the display 42, any of various types of displays can be used as appropriate. For example, a liquid crystal display (LCD), a cathode ray tube (CRT) display, an organic electroluminescence (OELD) display, or a plasma display can be used as the display 42. Furthermore, the display 42 may be provided on the gantry 10. In addition, the display 42 may be a desktop type or may be configured by a tablet terminal or the like capable of wirelessly communicating with the main body of the console 40.

The input interface 43 receives various input operations from an operator, converts the received input operations into electrical signals, and outputs the electrical signals to the processing circuitry 45. For example, the input interface 43 receives, from the operator, a collection condition for collecting count data, a reconstruction condition for reconstructing a PCCT image, an image processing condition for generating a post-processing image from a PCCT image, and the like. As the input interface 43, for example, a mouse, a keyboard, a trackball, a switch, a button, a joystick, a touch pad, a touch panel display, and the like can be appropriately used. Note that, in the present embodiment, the input interface 43 is not limited to one including physical operation components such as a mouse, a keyboard, a trackball, a switch, a button, a joystick, a touch pad, and a touch panel display. For example, an electric signal processing circuitry that receives an electric signal corresponding to an input operation from an external input device provided separately from the device and outputs the electric signal to the processing circuitry 45 is also included in the example of the input interface 43. Furthermore, the input interface 43 may be provided on the gantry 10. In addition, the input interface 43 may be configured by a tablet terminal or the like capable of wirelessly communicating with the main body of the console 40.

The communication interface 44 includes a network interface card (NIC) or the like for communicating various data with an external device such as a workstation, a picture archiving and communication system (PACS), a radiology information system (RIS), or a hospital information system (HIS) via a network.

The processing circuitry 45 controls the entire operation of the X-ray computed tomography apparatus 1 according to the electric signal of the input operation output from the input interface 43. The processing circuitry 45 includes a processor such as a CPU and a memory such as a ROM and a RAM as hardware resources. The processing circuitry 45 implements various functions by a processor that executes a program expanded in a memory. The various functions are not limited to being implemented by a single processing circuitry. A processing circuitry may be configured by combining a plurality of independent processors, and each processor may execute a program to implement various functions.

As shown in FIG. 1, the processing circuitry 45 implements a scan control function 51, an acquisition function 52, an image generation function 53, a storage function 54, and a display control function 55.

In the scan control function 51, the processing circuitry 45 controls the gantry 10 to perform the PCCT scan on the subject P. Under the control of the processing circuitry 45, the PCCT scan is performed by the gantry 10. With the PCCT scan performed, the data acquisition system 18 collects count data for each of the plurality of energy bins for each view.

In the acquisition function 52, the processing circuitry 45 acquires various types of information. As an example, the processing circuitry 45 acquires various kinds of examination information regarding the PCCT scan of a subject to be processed. The various kinds of examination information include at least generation purpose information and generation process information. The generation purpose information is information indicating a generation purpose of the PCCT image. The generation process information is information indicating a generation process of the PCCT image according to the generation purpose. It is assumed that the various kinds of examination information are set in advance in a hospital information system such as RIS or HIS, the X-ray computed tomography apparatus 1, or the like before the PCCT scan. In addition, the processing circuitry 45 may acquire count data collected by the PCCT scan from the data acquisition system 18.

In the image generation function 53, the processing circuitry 45 reconstructs the PCCT image corresponding to the generation purpose indicated by the generation purpose information acquired by the acquisition function 52 according to the generation process indicated by the generation process information acquired by the acquisition function 52 based on the count data collected by the PCCT scan of the subject to be processed. The PCCT image means an image based on the count data collected by the PCCT scan.

Here, the PCCT image can be applied to both a two-dimensional image related to one slice or slab and a three-dimensional image related to one volume. Note that a two-dimensional image means image data including a plurality of pixels (pixels) arranged in a two-dimensional space, and a three-dimensional image means image data including a plurality of pixels (voxels) arranged in a three-dimensional space. Hereinafter, it is assumed that the PCCT image is a three-dimensional image.

In the storage function 54, the processing circuitry 45 associates the various kinds of examination information acquired by the acquisition function 52 with the PCCT image generated by the image generation function 53, and stores the PCCT image in a storage device (hereinafter, a data storage device). Examples of the various kinds of examination information associated with the PCCT image include generation purpose information and generation process information. The data storage device may be the memory 41 provided in the X-ray computed tomography apparatus 1, or may be a storage device in an information system such as PACS, HIS, or RIS connected via the communication interface 44.

In the display control function 55, the processing circuitry 45 displays various types of information on the display 42. As an example, the processing circuitry 45 displays various kinds of examination information acquired by the acquisition function 52, the PCCT image generated by the image generation function 53, and the like. In this case, the processing circuitry 45 may display the generation purpose information and the generation process information together with the PCCT image. Note that the processing circuitry 45 converts the PCCT image into a visualized image and displays the visualized image on the display 42. As the conversion processing to the visualized image, conversion processing from a three-dimensional image to a two-dimensional image, such as a pixel value projection method such as maximum intensity projection (MIP), multi-planar reconstruction (MPR), volume rendering, or surface rendering, is used. Note that, in the following description, in order to avoid complication of the description, the visualized image and the PCCT image are not particularly distinguished, and are referred to as PCCT images.

Hereinafter, details of the X-ray computed tomography apparatus 1 will be described.

First, various kinds of examination information according to the present embodiment will be described. The various kinds of examination information include detector resolution information, bin setting information, generation purpose information, and generation process information.

The detector resolution information is information indicating the detector resolution, equivalent to the number of bundles of X-ray detection elements (the number of X-ray detection elements belonging to one acquisition channel of the electric signal). The data acquisition system 18 bundles the electrical signals from the plurality of X-ray detection elements for each acquisition channel and counts the bundled electrical signals. The detector resolution can be set to, for example, “SHR” representing ultra-high resolution or the number of bundles “1”, “HR” representing high resolution or the number of bundles “4”, or “NR” representing normal resolution or the number of bundles “9”.

The bin setting information means information about setting of energy bins, and specifically represents a quantity of energy bins and an energy range. The number of energy bins is not particularly limited as long as it is two or more. The energy range can be arbitrarily set for each energy bin.

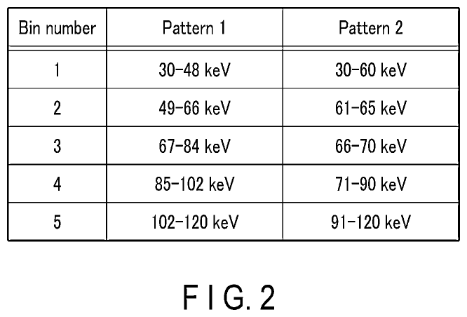

FIG. 2 is a diagram showing an example of setting the number of energy bins and the energy range. In the example of FIG. 2, it is assumed that the number of energy bins is five, and two patterns are shown for the energy range of each energy bin. Note that the tube voltage is 120 kVp, and it is assumed that noise is dominant in the energy range of 30 keV or less.

In pattern 1, the energy ranges of the five energy bins are set substantially evenly. Specifically, the energy range of the bin number “1” is “30 to 48 keV”, the energy range of the bin number “2” is “49 to 66 keV”, the energy range of the bin number “3” is “67 to 84 keV”, the energy range of the bin number “4” is “85 to 102 keV”, and the energy range of the bin number “5” is “102 to 120 keV”. Pattern 2 is a setting example for K-edge imaging, and it is assumed that the K absorption edge is near 65 to 66 keV. Specifically, the energy range of the bin number “1” is “30 to 60 keV”, the energy range of the bin number “2” is “61 to 65 keV”, the energy range of the bin number “3” is “66 to 70 keV”, the energy range of the bin number “4” is “71 to 90 keV”, and the energy range of the bin number “5” is “91 to 120 keV”.

The bin setting information may include a bin to be used, an energy representative value, and an image weighting coefficient in addition to the number of energy bins and the energy range. The bin to be used represents the energy bin used to generate the PCCT image. For example, if the PCCT image is generated using only the energy bin with the bin number “3” of pattern 1 of FIG. 2 above, the value of the bin to be used is “3”.

The energy representative value means an energy value representative of each energy bin and is used for energy integration. By multiplying the count data of the energy bin to be processed by the energy representative value of the energy bin, an energy integral value of the energy bin is obtained. The energy representative value may be set to a statistical value based only on the energy range, such as a median value or an average value of the energy range, or may be set to a value weighted by the energy spectrum of the X-ray emitted from the X-ray tube 11 or the X-ray detected by the X-ray detector 12 in the energy range. For example, “76 keV” may be set as the energy representative value of the bin number “3” of pattern 1 of FIG. 2.

The image weighting coefficient is a numerical value representing a degree of enhancement or suppression of each energy bin when generating a PCCT image using count data of a plurality of energy bins. The image weighting coefficient may be set according to the generation purpose or the like. For example, if the PCCT image is generated by evenly using all the energy bins of pattern 1 of FIG. 2, the image weighting coefficient “1” of the bin number “1”, the image weighting coefficient “1” of the bin number “2”, the image weighting coefficient “1” of the bin number “3”, the image weighting coefficient “1” of the bin number “4”, and the image weighting coefficient “1” of the bin number “5” are set. As another example, if the PCCT image enhancing a component on the low energy side such as fat is generated, the image weighting coefficient “1” of the bin number “1”, the image weighting coefficient “2” of the bin number “2”, the image weighting coefficient “2” of the bin number “3”, the image weighting coefficient “1/5” of the bin number “4”, and the image weighting coefficient “1/5” of the bin number “5” are set. In addition, the image weighting coefficient may be “0”.

The generation purpose information is information indicating the generation purpose of the PCCT image, in other words, the type of image a user desires to view. Examples of the generation purpose include an integral image, a basis material image, a virtual monochromatic image, and a material map. The generation purpose may be set in more detail. For example, in the case of the integral image, the number of a bin to be used, an energy range, or the like may be added, such as an energy bin “3” or an energy range “67 to 84 keV”. In the case of the basis material image or the material map, information of a material to be emphasized may be added, such as a basis material image or a material map of “bone”. For example, in the case of the virtual monochromatic image, a virtual monochromatic energy value or the like may be added, such as an energy value “74 keV”.

As described above, the generation process information is information indicating a generation process of the PCCT image according to the generation purpose. The generation process is expressed by a series of names and codes of processing to be performed in order to generate an image according to the generation purpose. The generation process is represented, as an example, by a combination of energy integral image reconstruction, material decomposition, material decomposition image reconstruction, virtual monochromatic image generation, and/or material map generation. Note that the generation process may be expressed in detail by adding bin setting information such as a bin to be used, an energy representative value, and an image weighting coefficient in addition to the name and code of each processing described above.

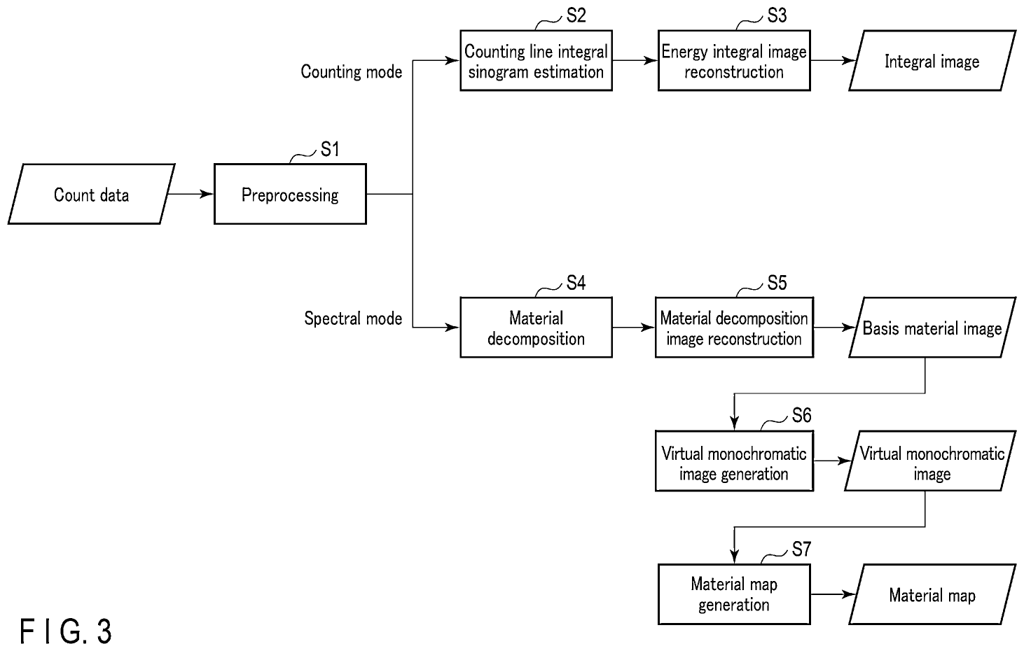

FIG. 3 is a diagram showing details of the process of generating the PCCT image. As shown in FIG. 3, the processing circuitry 45 performs preprocessing on the count data (step S1). As the preprocessing, data reformatting, beam hardening correction, and the like are performed. After step S1, the generation process is divided into two modes, a counting mode and a spectral mode. First, the counting mode will be described.

The processing circuitry 45 performs counting line-integral sinogram estimation on the preprocessed count data (step S2) to generate a counting line-integral sinogram. The counting line is an imaginary line reaching each X-ray detection element (or the center element of the acquisition channel) of the X-ray passing through the subject P, and corresponds to a projection line in the integrated CT. Upon step S2 being performed, the processing circuitry 45 performs energy integral image reconstruction on the counting line-integral sinogram (step S3), and reconstructs the integral image.

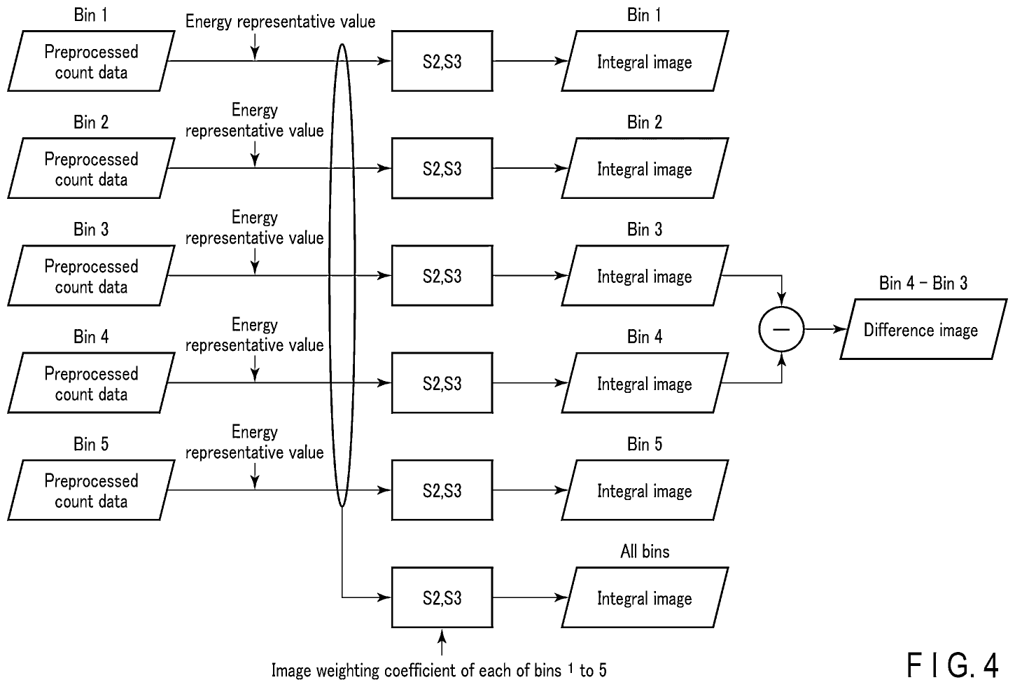

FIG. 4 is a diagram showing a specific example of a process of generating the counting mode. In FIG. 4, similarly to FIG. 2, it is assumed that the number of energies is five. As shown in FIG. 4, the processing circuitry 45 generates preprocessed count data of each of the energies 1 to 5. For example, the processing circuitry 45 may generate, for each energy bin, energy integral data by multiplying count data by an energy bin representative value of the energy bin for each counting line and summing over, perform counting line-integral sinogram estimation (S2) on the generated energy integral data, and perform filtered back projection (FBP) reconstruction on the counting line-integral sinogram to reconstruct an integral image of the energy bin (S3).

As another example, as shown in FIG. 4, the processing circuitry 45 may perform a counting line-integral sinogram estimation (S2) on the energy integral data for each of all of the energy bins, generate, for each of the energy bins, a weighted summed counting line-integral sinogram by multiplying the counting line-integral sinogram by an image weighting coefficient for that energy bin and summing over, and perform a filtered back projection (FBP) reconstruction on the weighted summed counting line-integral sinogram to reconstruct an integral image of all energy bins (S3). The values of the image weighting coefficients for the plurality of energy bins can be set arbitrarily.

As another example, as shown in FIG. 4, the processing circuitry 45 may generate a difference image between an integral image of energy bin “3” and an integral image of energy bin “4”. The energy bin of the difference target is not limited to the combination of “3” and “4”, and may be any energy bin.

Next, the spectral mode will be described with reference to FIG. 3. The processing circuitry 45 performs material decomposition on the preprocessed count data (step S4), and generates two basis material pathlength sinograms corresponding to two basis materials, respectively. The material decomposition can be performed in either the data domain or the image domain. The types of the two basis materials can be set to any type as generation purpose information and/or generation process information. As the type of the two basis materials, for example, a combination of bone and water, water and a contrast medium (iodine), and the like is possible. Note that the number of basis materials may be set to two or more and equal to or less than the total number of energy bins. Next, the processing circuitry 45 performs material decomposition image reconstruction on the two basis material pathlength sinograms (step S5), and reconstructs two basis material images corresponding to the two basis materials, respectively. The basis material image represents a spatial distribution of the corresponding basis material. The pixel value of the basis material image corresponds to the density value of the basis material and is different from the CT value.

As shown in FIG. 3, the processing circuitry 45 may perform virtual monochromatic image generation processing on the two basis material images (step S6) to generate a virtual monochromatic image corresponding to arbitrary X-ray energy (hereinafter, virtual monochromatic energy). The value of the virtual monochromatic energy can be set to any value as generation purpose information and/or generation process information. The pixel value of the virtual monochromatic image is a CT value.

As shown in FIG. 3, the processing circuitry 45 performs material map generation processing on the virtual monochromatic image (step S7), and generates two material maps corresponding to the two basis materials, respectively. The processing circuitry 45 may generate a composite image of two material maps. The pixel value of the material map is a CT value.

Next, a process procedure of a PCCT examination by the X-ray computed tomography apparatus 1 will be described.

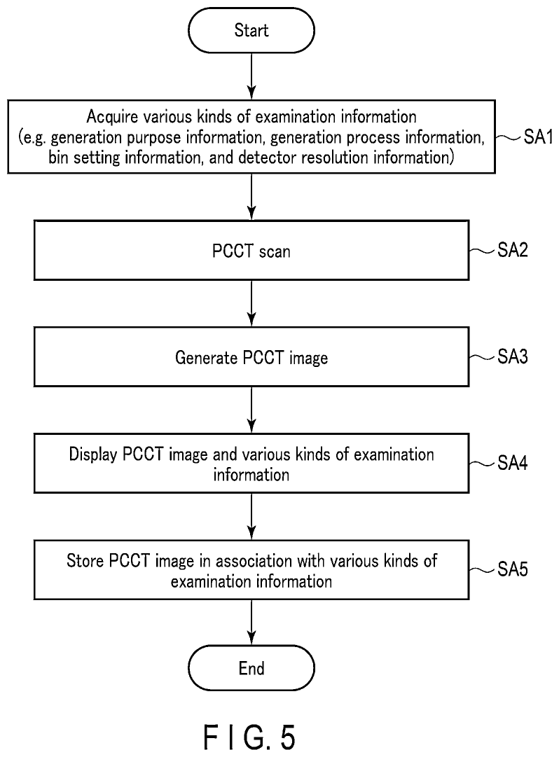

FIG. 5 is a view showing a process procedure of the PCCT examination by the X-ray computed tomography apparatus 1. As shown in FIG. 5, the processing circuitry 45 acquires various kinds of examination information by the acquisition function 52 (step SA1). As described above, the various kinds of examination information include the generation purpose information, the generation process information, the bin setting information, and the detector resolution information. It is assumed that the various kinds of examination information are set in advance at the time of ordering the PCCT examination by the RIS or the like. In this case, in step SA1, the processing circuitry 45 can acquire various kinds of examination information together with order information from the RIS that orders the PCCT examination. The various kinds of examination information can be set by another computer system, the processing circuitry 45 of the X-ray computed tomography apparatus 1, or the like. The acquired various kinds of examination information are stored in the memory 41.

Once step SA1 is performed, the processing circuitry 45 executes the PCCT scan by the scan control function 51 (step SA2). In step SA2, the processing circuitry 45 controls the gantry 10 according to a separately set scan condition to execute the PCCT scan on the subject P. Specifically, the number and value of the energy thresholds of the data acquisition system 18 are set according to the number and energy range of the energy bins included in the bin setting information. In addition, the detector resolution of the data acquisition system 18 is set according to the detector resolution included in the detector resolution information.

Once step SA2 is performed, the processing circuitry 45 generates a PCCT image based on the count data collected in step SA2 (step SA3). In step SA3, the processing circuitry 45 generates a PCCT image matching the generation purpose information and the generation process information. Specifically, the processing circuitry 45 sequentially processes the count data according to the generation process indicated by the generation process information to generate the PCCT image corresponding to the generation purpose indicated by the generation purpose information.

Once step SA3 is performed, the processing circuitry 45 displays the PCCT image generated in step SA3 and the various kinds of examination information acquired in step S1 (step SA4). Various methods can be performed as a method of displaying the PCCT image and various kinds of examination information. As an example, the processing circuitry 45 superimposes and displays text indicating various kinds of examination information on the PCCT image. The type of various kinds of examination information to be displayed can be arbitrarily selected via the input interface 43 or the like. The text indicating the various kinds of examination information may be set in advance by the user or the like, or may be converted from the various kinds of examination information according to any automatic conversion algorithm.

Hereinafter, a specific example of display of the PCCT image and various kinds of examination information will be described. Here, a case of generating a PCCT image with a generation purpose “equivalent to 75 keV” will be considered. As a process of generating the PCCT image, for example, as shown in FIGS. 3 and 4, pattern 1 for generating an integral image of the third energy bin equivalent to the energy range “70 to 80 keV” and pattern 2 for generating a virtual monochromatic image of the virtual monochromatic energy “75 keV” are considered. More specifically, pattern 1 undergoes a process of “performing energy integral image reconstruction to generate an integral image using count data of energy bin “3” ”. More specifically, pattern 2 undergoes a process of “performing material decomposition using all count data in the energy bins “1” to “5”, performing basis material image reconstruction to generate two basis material images corresponding to the two basis materials, respectively, and performing virtual monochromatic image generation processing to generate a virtual monochromatic image”.

In a case where such two PCCT images having the same generation purpose and different generation processes are displayed without being particularly distinguished from each other, it is likely difficult to know which generation process each image is generated through. In order to reduce such an adverse effect, the processing circuitry 45 displays a generation purpose and a generation process together with each image.

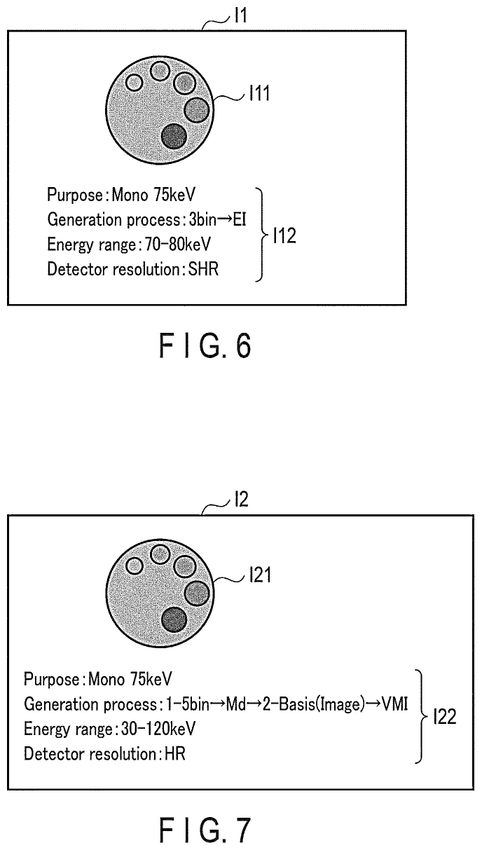

FIG. 6 is a diagram showing an example of a display screen I1 for a PCCT image I11 with the generation purpose “equivalent to 75 keV” according to pattern 1 described above and various kinds of examination information 112. As described above, in FIG. 6, the generation purpose “equivalent to 75 keV” is represented by the text “Mono 75 keV”. As described above, pattern 1 undergoes a process of “performing energy integral image reconstruction to generate an integral image using count data of energy bin “3” ”, and is represented by the text “3bins→EI” in FIG. 6. “3bin” means to use count data for the energy bin “3”. “EI” means energy integration (EI) image reconstruction. In addition, as shown in FIG. 6, the text “70-80 keV” indicating the energy range, the text “SHR” indicating the detector resolution, and the like may be displayed.

FIG. 7 is a diagram showing an example of a display screen 12 for a PCCT image 121 with the generation purpose “equivalent to 75 keV” according to pattern 2 described above and various kinds of examination information 122. As described above, in FIG. 7, the generation purpose “equivalent to 75 keV” is represented by the text “Mono 75 keV”. As described above, pattern 2 undergoes a process of “performing material decomposition using all count data in the energy bins “1” to “5”, performing basis material image reconstruction to generate two basis material images corresponding to the two basis materials, respectively, and performing virtual monochromatic image generation processing to generate a virtual monochromatic image”, and is represented by the text “1-5bin→Md→2-Basis(Image)→VMI” in FIG. 7. “1-5bin” means to use count data for the energy bins “1” to “5”. “Md” means material decomposition (MD), “2-Basis (Image)” means reconstruction processing for generating two basis material images corresponding to two basis materials, respectively, and “VMI” means reconstruction processing for a virtual monochromatic image. In addition, as shown in FIG. 7, a text “30-120 keV” indicating the energy range, a text “HR” indicating the detector resolution, and the like may be displayed.

As shown in FIGS. 6 and 7, the processing circuitry 45 displays text indicating a generation purpose and text indicating a generation process on each of the PCCT images I11 and I21. Both of the texts may be displayed in a margin portion of the display screens I1 and 12 other than the PCCT images I11 and I21. As can be seen by comparing FIGS. 6 and 7, even if the generation purpose “Mono 75 keV” is the same, the generation process and eventually the characteristics of the PCCT image can be grasped by displaying the text indicating the generation process together with the PCCT image. For example, by checking the generation process of the image in FIG. 6, it can be seen that the display image is an integral image corresponding to the third energy bin, and by checking the generation process of the image in FIG. 7, it can be seen that the display image is a virtual monochromatic image generated from the basis material image. As described above, in a case where there are a plurality of PCCT images having the same generation purpose and different generation processes, by displaying the generation process together with the PCCT image, it is possible to reduce the possibility that the users will confuse their own PCCT images with each other.

Another specific example will be described. It is assumed that the generation purpose according to this specific example is “material decomposition (iodine & calcium)”. The generation purpose means a PCCT image using iodine and calcium as basis materials. Such a PCCT image may correspond to a basis material image or a material map.

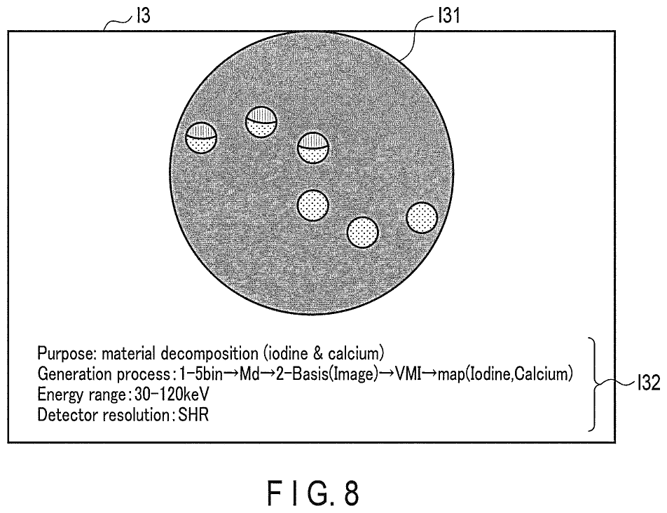

FIG. 8 is a diagram showing a display example of a PCCT image of a generation purpose “material decomposition (iodine & calcium)” and various kinds of examination information. In FIG. 8, a material map (hereinafter, an iodine & calcium map) representing the spatial distribution of the CT values of the iodine and calcium map is displayed as the PCCT image of the generation purpose “material decomposition (iodine & calcium)”. A composite image of the material map of iodine and the material map of calcium (bone) is displayed. In an example of the iodine & calcium map generation procedure, first, the processing circuitry 45 performs threshold processing on the iodine material map to extract an iodine region, performs threshold processing on the calcium material map to extract a calcium region, and generates the iodine & calcium map by combining the iodine region (the hatched portion in FIG. 8) and the calcium region (the dot hatched portion in FIG. 8).

The generation process shown in FIG. 8 undergoes a process of “performing material decomposition using all count data in the energy bins “1” to “5”, performing basis material image reconstruction to generate two basis material images corresponding to the two basis materials, respectively, performing virtual monochromatic image generation processing to generate a virtual monochromatic image, and performing the material map generation processing to generate the iodine & calcium material map”, and is represented by the text “1-5bin→Md→2-Basis(Image)→VMI→map (Iodine, Calcium)” in FIG. 8. “1-5bin” means to use count data for the energy bins “1” to “5”. “Md” means material decomposition (MD), “2-Basis (Image)” means reconstruction processing for two basis material images corresponding to two basis materials, respectively, and “VMI” means reconstruction for a virtual monochromatic image. “map (Iodine, Calcium)” means a process of generating a material map of iodine and calcium. In addition, as shown in FIG. 8, the text “30-120 keV” indicating the energy range, the text “SHR” indicating the detector resolution, and the like may be displayed. As shown in FIG. 8, it is possible to avoid confusion with the basis material image by displaying the generation process together with the material map.

In the examples of FIGS. 6 to 8, items of various kinds of examination information such as a generation purpose, a generation process, an energy range, and a detector resolution are displayed. However, the present embodiment is not limited thereto. The processing circuitry 45 may switch display or non-display of each item included in the various kinds of examination information according to the user's instruction or a predetermined algorithm. In the PCCT images I11, I21, and 131 of FIGS. 6 to 8, the phantom is the subject, but can be appropriately replaced with an image in which the subject P is the subject.

Once step SA4 is performed, the processing circuitry 45 associates the PCCT image generated in step SA3 with the various kinds of examination information acquired in step S1, and stores the PCCT image in the data storage device (step SA5). The processing circuitry 45 may associate all the items of the various kinds of examination information, or may associate only some items of all the items of the various kinds of examination information. The item to be associated can be arbitrarily selected according to the user's instruction or a predetermined algorithm.

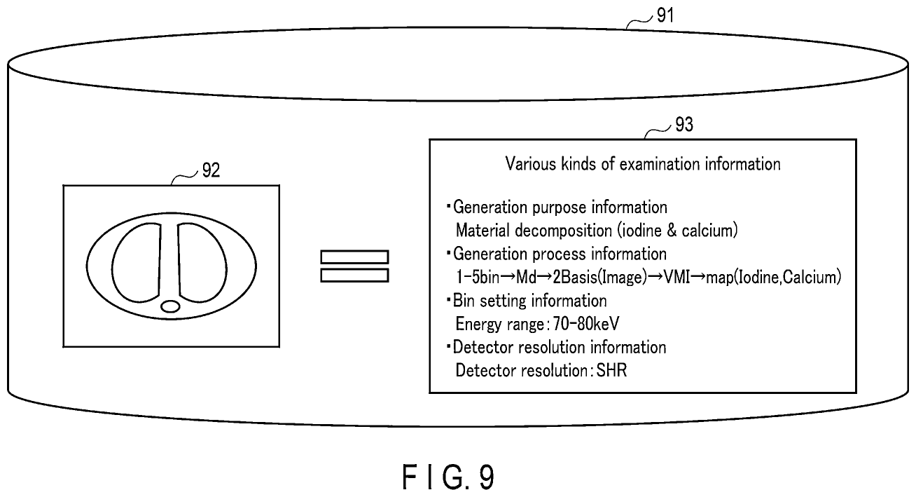

FIG. 9 is a conceptual diagram of association between a PCCT image 92 and various kinds of examination information 93. As shown in FIG. 9, the processing circuitry 45 associates the PCCT image 92 generated in step SA3 with the various kinds of examination information 93 acquired in step SA1, and stores the PCCT image 92 and the various kinds of examination information 93 that are associated with each other in the data storage device 91. The various kinds of examination information 93 may be associated with text information indicating the generation purpose information, the generation process information, the bin setting information, and the detector resolution information. By storing the PCCT image 92 and the various kinds of examination information 93 in association with each other, it is possible to read and confirm a generation purpose, a generation process, and the like of the PCCT image 92 at any timing. Therefore, it is possible to easily determine a generation purpose, a generation process, and the like of the PCCT image 92.

Various methods can be selected as a method of associating the PCCT image with various kinds of examination information. Hereinafter, an association method will be exemplified. The association method is not limited to the following method.

<Method 1> The processing circuitry 45 may associate the PCCT image with various kinds of examination information by inputting various kinds of examination information into a comment input field of the PCCT image. The comment input field means a graphical user interface (GUI) component superimposed on the PCCT image. For example, generation purpose information may be input to an image comment 1, generation process information may be input to an image comment 2, bin setting information may be input to an image comment 3, and detector resolution information may be input to an image comment 4. The text input in the comment input field is integrally displayed as one element of the PCCT image.

<Method 2> The processing circuitry 45 may associate the PCCT image with various kinds of examination information by inputting various kinds of examination information into a data element corresponding to a DICOM standard tag of the PCCT image.

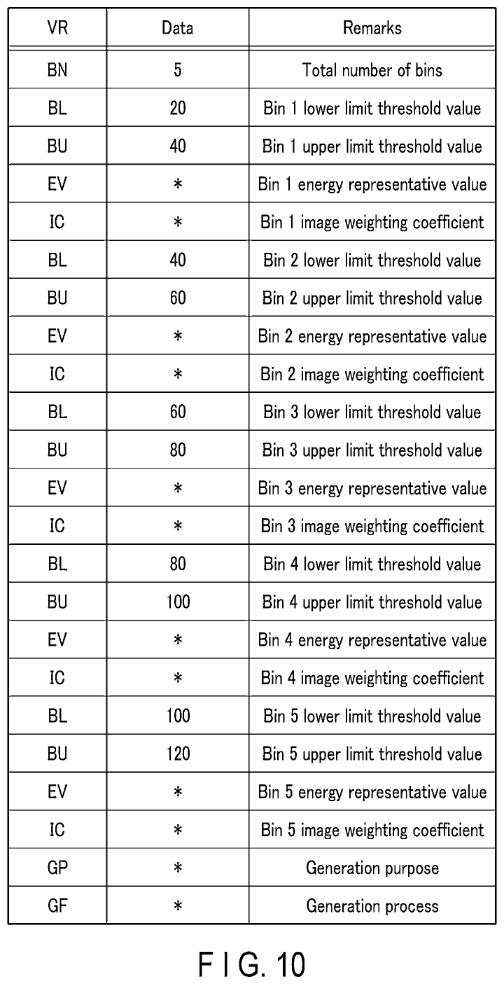

FIG. 10 is a diagram showing a first example of input of various kinds of examination information to the DICOM standard tag. FIG. 10 shows a data representation method (Value Representation (VR)) of the DICOM standard tag, data, and remarks column. As the bin setting information, the total number of bins, the lower limit threshold value, the upper limit threshold value, the energy bin representative value, and the image weighting coefficient are input. In the first example input, a lower threshold value, an upper threshold value, an energy bin representative value, and an image weighting coefficient are input with different DICOM tags for each energy bin. For example, the data for the lower threshold value (VR=“BL”) of the energy bin “2” is “40”, the data for the upper threshold value (VR=“BU”) is “60”, the data for the energy representative value (VR=“EV”) is *”, and the data for the image weighting coefficient (VR=“IC”) is “*”. The generation purpose is input as the generation purpose information. VR of the generation purpose may be defined as “GP” or the like, and text or the like indicating the generation purpose may be input to the data. The generation process is input as the generation process information. VR of the generation process may be defined as “GF” or the like, and text or the like indicating the generation process may be input to the data.

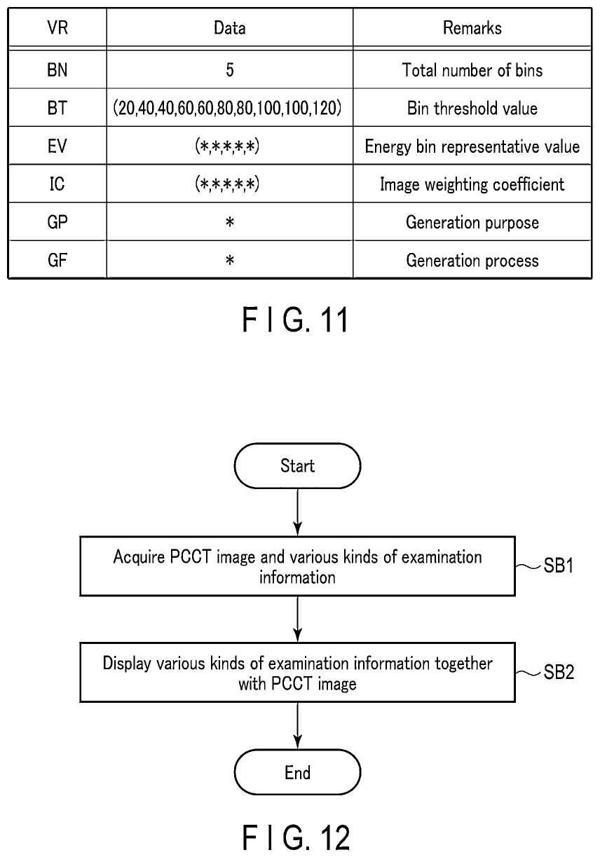

FIG. 11 is a diagram showing a second example of input of various kinds of examination information to the DICOM standard tag. FIG. 11 shows, as in FIG. 10, a data representation method of the DICOM standard tag, data, and remarks column. In the second example input, a threshold for an energy bin, an energy bin representative value, and an image weighting coefficient are input with a DICOM tag common to all energy bins. Note that VR of the threshold is defined by “BT” or the like, and the lower limit threshold and the upper limit threshold are arranged in the data according to the order of the energy bins. Similarly, in the data of the energy bin representative value and the image weighting coefficient, the data values are arranged according to the order of the energy bins. For example, data for the threshold value (VR=“BT”) is “20, 40, 40, 60, 60, 80, 80, 100, 100, 120”, data for the energy bin representative value (VR=“EV”) is “*,*,*,*,*, and data for the image weighting coefficient (VR=“IC”) is “*,*,*,*,*,*”.

Note that the input examples of FIGS. 10 and 11 are examples, and information other than the information shown in FIGS. 10 and 11 may be input. Furthermore, the value of VR is also an example, and is not limited thereto, and may be defined by any character string.

<Method 3> The processing circuitry 45 may input an association between the PCCT image and various kinds of examination information into a data element corresponding to a DICOM private tag of the PCCT image. The VR of the DICOM private tag corresponding to the item of various kinds of examination information can be arbitrarily set by the user.

Once step SA5 is performed, the PCCT examination according to the present embodiment ends.

Note that the processing procedure of the PCCT examination shown in FIG. 5 is an example, and the present embodiment is not limited thereto. For example, the order of steps SA4 and SA5 may be reversed. In addition, either one of steps SA4 and SA5 may be omitted. Step SA1 may be performed between step SA2 and step SA3.

The present embodiment is not limited to the above embodiment, and any element can be deleted, added, and/or changed without departing from the gist of the invention.

### First Modification

In the above embodiment, the medical image processing apparatus 40 is the console provided in the X-ray computed tomography apparatus 1. However, the present embodiment is not limited thereto. The medical image processing apparatus 40 according to the first modification may be a computer separate from the X-ray computed tomography apparatus 1 such as an image interpretation apparatus, image examination apparatus, or a workstation. In this case, the medical image processing apparatus 40 does not need to include the scan control function 51. The processing circuitry 45 according to the first modification acquires the PCCT image and the various kinds of examination information from the data storage device, and displays the acquired various kinds of examination information together with the PCCT image.

FIG. 12 is a diagram showing a process procedure of display processing of the PCCT image by the medical image processing apparatus 40 according to the first modification. In addition, it is assumed that the PCCT image and various kinds of examination information are stored in the data storage device before the start of FIG. 12.

As shown in FIG. 12, first, the processing circuitry 45 acquires a PCCT image to be displayed and various kinds of examination information associated with the PCCT image from the data storage device by the acquisition function 52 (step SB1). The PCCT image to be displayed can be arbitrarily designated by the user via the input interface 43 or the like.

Once step SB1 is performed, the processing circuitry 45 displays various kinds of examination information on the display 42 together with the PCCT image acquired in step SB1 by the display control function 55 (step SB2). In step SB2, as shown in FIGS. 6, 7, and 8, the processing circuitry 45 may superimpose text indicating various kinds of examination information on the PCCT image. As a result, it is possible to immediately display and confirm various kinds of examination information such as a generation purpose and a generation process of the PCCT image without confusion with other PCCT images.

A method of displaying the various kinds of examination information is different according to a method of associating the PCCT image with the various kinds of examination information. For example, in the case of <Method 1>, since the text of various kinds of examination information is input in the comment input field of the PCCT image, the text of various kinds of examination information is displayed as one element of the PCCT image. In the case of <Method 2>, the processing circuitry 45 reads text of various kinds of examination information from the DICOM standard tag, and superimposes and displays the read text on the PCCT image. In the case of <Method 3>, as in <Method 2>, the processing circuitry 45 reads text of various kinds of examination information from the DICOM private tag, and superimposes and displays the read text on the PCCT image.

Once step SB2 is performed, the display processing according to the first modification shown in FIG. 12 ends.

According to the first modification, in the medical image processing apparatus 40 independent of the X-ray computed tomography apparatus 1, similarly to the above embodiment, the PCCT image and various kinds of examination information can be stored in association with each other, and various kinds of examination information can be displayed together with the PCCT image. As a result, it is possible to easily grasp a generation purpose and a generation process of the PCCT image.

### Second Modification

The processing circuitry 45 according to the second modification may associate an intermediate image generated in the generation process of the PCCT image corresponding to the generation purpose (hereinafter, the generation purpose image) with the examination information including the generation process information corresponding to the intermediate image, and store the intermediate image in the data storage device. For example, in the case of FIG. 7, a material decomposition image that is an intermediate image is generated in the process of generating a virtual monochromatic image that is the generation purpose image. The processing circuitry 45 according to the second modification stores the same generation process information as the generation process information of the virtual monochromatic image in the data storage device in association with the material decomposition image. Specifically, as the generation process information, the same text “1-5bin→Md→2-Basis(Image)→VMI” as the generation process of the virtual monochromatic image is associated with the material decomposition image as the intermediate image.

Note that the processing circuitry 45 may store generation process information of an intermediate image in association with the intermediate image. The processing circuitry 45 can generate the generation process information of the intermediate image by extracting a portion corresponding to the intermediate image generation process in the generation process of the generation purpose image. For example, in the case of FIG. 7, it is possible to extract the text “1-5bin→Md→2-Basis(Image)” in the generation process of the material decomposition image as the intermediate image from the text “1-5bin→Md→2-Basis(Image)→VMI” in the generation process of the virtual monochromatic image as the generation purpose image.

The processing circuitry 45 may store examination information other than the generation process information, such as generation purpose information, bin setting information, and detector resolution information, in association with the intermediate image. In this case, as the generation purpose information, the bin setting information, and the detector resolution information, the same information as the generation purpose image may be used.

### Third Modification

In the above embodiment, the generation purpose information and the generation process information are determined before the PCCT scan is performed. However, the present embodiment is not limited thereto. The processing circuitry 45 according to the third modification may determine the generation purpose information and the generation process information after performing the PCCT scan. For example, the generation purpose information and the generation process information may be determined for the first time only after the execution of the PCCT scan, or first generation purpose information and generation process information may be determined before or after the execution of the PCCT scan, and then the second generation purpose information different from the original purpose and the generation process information corresponding to the second generation purpose information may be determined. The processing circuitry 45 according to the third modification can also store and/or display the examination information including the second generation purpose information and the generation process information in association with the PCCT image generated according to the second generation purpose information and the generation process information.

In the above embodiment, the generation purpose is, for example, an integral image, a basis material image, a virtual monochromatic image, and a material map. However, the present embodiment is not limited thereto. The generation purpose according to the present embodiment may be an electron density image, an effective atomic number image, a K-edge image, or any other image that can be generated in the PCCT.

### Fourth Modification

The X-ray computed tomography apparatus 1 according to the fourth modification executes the PCCT scan, generates the PCCT image, and/or displays the PCCT image according to the various kinds of examination information stored in step SA5. Hereinafter, the X-ray computed tomography apparatus 1 according to the fourth modification will be described.

Similarly to the first modification, the processing circuitry 45 according to the fourth modification acquires a PCCT image to be displayed and the various kinds of examination information associated with the PCCT image from the data storage device by the acquisition function 52, and displays the various kinds of examination information on the display 42 together with the acquired PCCT image by the display control function 55. The PCCT image to be displayed can be arbitrarily designated by the user via the input interface 43 or the like.

Here, it is assumed that the user who has confirmed the PCCT image and the various kinds of examination information desires to perform the PCCT scan and the image generation under the same condition as the PCCT image.

Specifically, if the processing circuitry 45 receives an instruction to execute the same scan as the acquired various kinds of examination information of the past scan, the processing circuitry 45 controls the gantry 10 according to the examination information by the scan control function 51 to execute another PCCT scan (current scan). Specifically, the energy bin and the detector resolution of the X-ray detector 12 for the current scan are set according to the bin setting information and the detector resolution information in the various kinds of examination information of the past scan, and the current scan is performed under the set energy bin and detector resolution. This makes it possible to perform the current scan with the same energy bin and detector resolution as in the past scan.

The processing circuitry 45 generates, according to the generation process, another PCCT image (current image) according to the generation purpose among the various kinds of examination information of the previous scan based on the count data collected by the current scan by the image generation function 53. Then, the processing circuitry 45 displays the current image on the display 42 by the display control function 55. For example, in a case where the generation purpose of the previous scan is “equivalent to 75 keV” and the generation process is “1-5bin→Md→2-Basis(Image)→VMI”, the processing circuitry 45 performs material decomposition on the count data of the energy bins 1 to 5, generates two basis material images corresponding to the two basis materials, respectively, and reconstructs a virtual monochromatic image. As a result, it is possible to generate and display the current image according to the same generation purpose and generation process as the generation purpose and generation process of the past scan.

According to the fourth modification, by diverting various kinds of examination information of the past scan stored in association with the PCCT image to the current scan, the PCCT scan and the image generation under the same conditions as those of the past scan can be easily executed.

### CONCLUSION

According to some embodiments described above, the medical image processing apparatus 40 includes the processing circuitry 45 that implements the acquisition function 52, the image generation function 53, and the storage function 54. The processing circuitry 45 acquires examination information that is related to the PCCT scan and includes generation purpose information indicating a generation purpose of an image and generation process information indicating a generation process of the image according to the generation purpose. The processing circuitry 45 generates, according to the generation process, a PCCT image according to the generation purpose based on the count data collected by the PCCT scan. The processing circuitry 45 stores the examination information including the generation purpose information and the generation process information in a data storage device in association with the PCCT image.

According to other embodiments, the medical image processing apparatus 40 includes the processing circuitry 45 that implements the acquisition function 52, the image generation function 53, and the display control function 55. The processing circuitry 45 acquires examination information that is related to the PCCT scan and includes generation purpose information indicating a generation purpose of an image and generation process information indicating a generation process of the image according to the generation purpose. The processing circuitry 45 generates, according to the generation process, a PCCT image according to the generation purpose based on the count data collected by the PCCT scan. The processing circuitry 45 displays the examination information including the generation purpose information and the generation process information on the display 42 together with the PCCT image.

According to at least one of the embodiments described above, it is possible to easily determine the generation purpose and the generation process of the PCCT image.

The term “processor” used in the above description means, for example, a CPU, a GPU, or a circuit such as an application specific integrated circuit (ASIC) or a programmable logic device (for example, a simple programmable logic device (SPLD), a complex programmable logic device (CPLD), and a field programmable gate array (FPGA)). The processor implements a function by reading and executing a program stored in the storage circuit. Instead of storing the program in the storage circuit, the program may be directly incorporated in the circuit of the processor. In this case, the processor implements the function by reading and executing the program incorporated in the circuit. On the other hand, in a case where the processor is, for example, an ASIC, the function is directly incorporated as a logic circuit in a circuit of the processor instead of storing the program in the storage circuit. Note that each processor of the present embodiment is not limited to a case where each processor is configured as a single circuit, and a plurality of independent circuits may be combined and configured as one processor to implement the function. Furthermore, a plurality of components in FIG. 1 may be integrated into one processor to implement the function.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A medical image processing apparatus comprising a processing circuitry configured to:
acquire examination information related to a PCCT scan, the examination information including generation purpose information indicating a generation purpose of an image and generation process information indicating a generation process of the image according to the generation purpose;
generate, according to the generation process, a PCCT image according to the generation purpose based on count data collected by the PCCT scan; and
store the examination information including the generation purpose information and the generation process information in a storage device in association with the PCCT image.

2. The medical image processing apparatus according to claim 1, wherein the generation process is represented by a combination of energy integral image reconstruction, material decomposition, material decomposition image reconstruction, virtual monochromatic image generation, and/or material map generation.

3. The medical image processing apparatus according to claim 1, wherein
the processing circuitry is configured to:
further acquire bin setting information indicating the number of energy bins and/or energy range in the PCCT scan; and
further associate the bin setting information with the PCCT image.

4. The medical image processing apparatus according to claim 1, wherein the processing circuitry is configured to display the examination information including the generation purpose information and the generation process information on a display device together with the PCCT image.

5. The medical image processing apparatus according to claim 4, wherein
the processing circuitry is configured to:
acquire the PCCT image and the examination information from the storage device; and
display the acquired examination information together with the PCCT image.

6. The medical image processing apparatus according to claim 4, wherein the processing circuitry is configured to superimpose and display text indicating the generation purpose information and text indicating the generation process information on the PCCT image.

7. The medical image processing apparatus according to claim 4, wherein the processing circuitry is configured to switch display or non-display of each item included in the examination information according to a user's instruction or a predetermined algorithm.

8. The medical image processing apparatus according to claim 1, wherein the processing circuitry is configured to input the examination information in a comment input field of the PCCT image reconstructed.

9. The medical image processing apparatus according to claim 1, wherein the processing circuitry is configured to input the examination information in a data element corresponding to a DICOM standard tag of the PCCT image reconstructed.

10. The medical image processing apparatus according to claim 1, wherein the processing circuitry is configured to input the examination information in a data element corresponding to a DICOM private tag of the PCCT image reconstructed.

11. The medical image processing apparatus according to claim 1, wherein the processing circuitry is configured to associate an intermediate image generated in the generation process of the PCCT image with other generation process information corresponding to the intermediate image and store the intermediate image in the storage device.

12. A medical image processing apparatus comprising a processing circuitry configured to:
acquire examination information related to a PCCT scan, the examination information including generation purpose information indicating a generation purpose of an image and generation process information indicating a generation process of the image according to the generation purpose;
generate, according to the generation process, a PCCT image according to the generation purpose based on count data collected by the PCCT scan; and
display the examination information including the generation purpose information and the generation process information on a display device together with the PCCT image.

13. An X-ray computed tomography apparatus comprising:
a gantry including an X-ray tube that generates an X-ray, an X-ray detector that detects the X-ray generated from the X-ray tube, and a data acquisition system that collects count data of the X-ray detected by the X-ray detector; and
a processing circuitry wherein
the processing circuitry is configured to:
acquire examination information related to a PCCT scan, the examination information including generation purpose information indicating a generation purpose of an image and generation process information indicating a generation process of the image according to the generation purpose;
generate, according to the generation process, a PCCT image according to the generation purpose based on count data collected by the data acquisition system; and
store the examination information including the generation purpose information and the generation process information in a storage device in association with the PCCT image.

14. The X-ray computed tomography apparatus according to claim 13, wherein
the processing circuitry is configured to:
acquire the PCCT image and the examination information from the storage device;
display the acquired examination information on a display device together with the PCCT image; and
in a case of receiving an instruction to execute the same scan as the acquired examination information, control the gantry according to the acquired examination information to execute another PCCT scan.

15. The X-ray computed tomography apparatus according to claim 14, wherein the processing circuitry is configured to generate another PCCT image according to the generation purpose in the acquired examination information according to the generation process based on count data collected by the other PCCT scan.

16. The X-ray computed tomography apparatus according to claim 15, wherein the processing circuitry is configured to display the other PCCT image on a display device.
